(19) United States
(12) Patent Application Publication
(10) Pub. No.: US 2026/0175042 A1
(43) Pub. Date: Jun. 25, 2026

(54) LASER STIMULATION AND MANAGEMENT SYSTEM

(71) Applicant: New Advantage Marketing Business Co., Ltd., Taipei City (TW)

(72) Inventor: Rudy Peng, Taipei City (TW)

(21) Appl. No.: 19/420,131

(22) Filed: Dec. 15, 2025

(30) Foreign Application Priority Data
Dec. 20, 2024 (TW) 113150037

(51) Int. Cl. A61N 5/06; A61N 5/067

(52) U.S. Cl. CPC A61N 5/0619; A61N 5/067; A61N 2005/0626

(57) ABSTRACT

A laser stimulation and management system comprises a control device connected with a server and a laser device through a communicating module. The server stores a user’s information, a customized massage setting information and a parameter table of a massage physiological data corresponding to the user’s information. The control device stores at least one massage setting parameter. When the server receives a physiological data information uploaded from the control device, the server updates the customized massage setting information according to the physiological data information and the parameter table, and transmits the updated customized massage setting information to the control device. The control device updates the at least one massage setting parameter according to the customized massage setting information and controls the laser device to generate a customized stimulating laser beam.

### CROSS REFERENCE TO RELATED APPLICATIONS

The present application claims priority to Taiwan application No. 113150037, filed on December 20, 2024, the content of which is hereby incorporated by reference in its entirety.

### BACKGROUND OF THE INVENTION

### 1. Field of the Invention

The present invention relates to a laser stimulation and management system that can intelligently assist in managing data for stimulating acupuncture points of a user from a laser device.

### 2. Description of the Related Art

In the popular understanding of folk medicine and health preservation, applying pressure to acupuncture points is considered as an excellent way to promote human health. Currently, there are already massage electronic products on the market that combine technology with folk health concepts, such as a laser massager that emits a laser massage beam by a laser emitter. The laser massager can generate laser massage beams with specific wavelengths to stimulate acupuncture points on a human body, thereby being helpful for producing benefits for the human body under concepts of folk medicine and health preservation.

However, the conventional laser massager only has a limited number of built-in laser stimulation options and cannot be appropriately customized for users with different physiologies. Users of the conventional laser massager may have different physiological structures and conditions, such as age, weight, gender, height, etc. This means that the conventional laser massager cannot provide appropriate laser massage tailored to unique physiological structures and conditions of each user, which may result in some users failing to have a comfortable massage experience. For example, a massage duration is too long for elder users, a massage pressure is too weak for users with strong physique; these are reasons why some users may not have the comfortable massage experience. Therefore, since built-in parameters of the conventional laser massager cannot be properly customizable to physiological data of a user, the conventional laser massager cannot fully meet the massage needs of the users.

### SUMMARY OF THE INVENTION

In view of the abovementioned problems, the present invention provides a laser stimulation and management system that can intelligently assist in managing data for stimulating acupuncture points of a user from a laser device connected with a control device, and use the managed data to update setting parameters of the laser device. The laser device therefore generates a more appropriate stimulating laser beam to massage the user.

The laser stimulation and management system, comprises:

a laser device comprising:

a laser emitter generating and emitting a stimulating laser beam; and

a communicating and processing module electrically connected with the laser emitter;

a server storing a user’s information, a customized massage setting information corresponding to the user’s information, and a parameter table of a massage physiological data corresponding to the user’s information; and

a control device comprising:

a communicating module communicatively connected with the communicating and processing module of the laser device and communicatively connected with the server through a network;

a memory module storing at least one massage setting parameter;

a display module;

an input module; and

a processing module electrically connected with the communicating module, the memory module, the display module, and the input module;

wherein when the processing module receives a physiological data information from the input module, the processing module transmits the physiological data information to the server through the communicating module;

wherein when the server receives the physiological data information, the server updates the customized massage setting information according to the physiological data information and the parameter table, and transmits an updated customized massage setting information to the processing module;

wherein when the processing module receives the updated customized massage setting information from the communicating module, the processing module updates the at least one massage setting parameter according to the customized massage setting information and controls the laser emitter of the laser device through the communicating module to generate the stimulating laser beam according to the at least one updated massage setting parameter;

wherein the customized massage setting information updated by the server comprises a massage acupuncture points information, and the at least one massage setting parameter stored in the memory module of the control device comprises a massage-acupuncture-point type;

wherein after the server transmits the updated customized massage setting information to the processing module, the processing module of the control device updates the massage-acupuncture-point type stored in the memory module according to the massage acupuncture points information;

wherein the processing module of the control device controls the display module to display an acupuncture point position information corresponding to the massage-acupuncture-point type.

The server assists in managing the physiological data information uploaded by the control device and updates the customized massage setting information according to the physiological data information and the parameter table, and transmits the updated customized massage setting information to the control device. A user can use the control device with a more appropriate and more personalized setting to control the laser device to provide a massage. That is, the processing module of the control device updates the at least one massage setting parameter according to the customized massage setting information, and controls the laser emitter of the laser device to generate the stimulating laser beam according to the at least one massage setting parameter.

In practical use, the user can input the user’s physiological data in advance through the input module of the control device before the massage, such as a height, a weight, a medical history, etc. Therefore, the input module of the control device generates the physiological data information to the processing module, and then the processing module uploads the physiological data information to the server through the communicating module to update the information of the user stored in the server. When the user starts being massaged, the server can load the latest and suitable parameter for the user to the laser device, so that the laser device generates the most suitable stimulation laser beam for the user.

Please note that the laser device is not a medical device and is merely a massage device used for folk medicine and health preservation. The parameters for generating the stimulating laser beam of the laser device of the present invention can be appropriately modified. Therefore, compared with the conventional laser device, the laser device of the present invention can generate the stimulating laser beam to the user, wherein the stimulating laser beam is more comfortable, more suitable, and more health-preservation enhancing for the user.

### BRIEF DESCRIPTION OF THE DRAWINGS

FIG. 1 is a block diagram of a laser stimulation and management system of the present invention;

FIG. 2 is a schematic diagram of connection of the laser stimulation and management system of the present invention;

FIG. 3 is a flow chart of an embodiment of a laser stimulation and management method executed by the laser stimulation and management system of the present invention;

FIG. 4 is a flow chart of another embodiment of the laser stimulation and management method executed by laser stimulation and management system of the present invention; and

FIG. 5 is a flow chart of another embodiment of the laser stimulation and management method executed by laser stimulation and management system of the present invention.

### DETAILED DESCRIPTION OF THE INVENTION

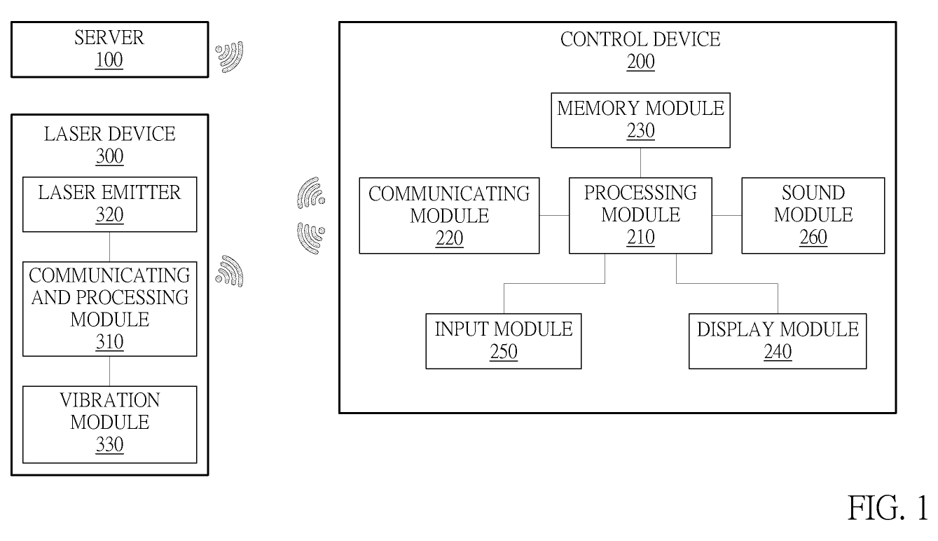

Referring to FIG. 1, the present invention provides a laser stimulation and management system. The laser stimulation and management system comprises a server 100, a control device 200, and a laser device 300. In the present embodiment, the control device 200 is a smart device, such as a smartphone or a tablet computer.

The server 100 stores a user’s information, a customized massage setting information corresponding to the user’s information, and a parameter table of a massage physiological data corresponding to the user’s information.

The laser device 300 comprises a communicating and processing module 310, and a laser emitter 320. The communicating and processing module 310 is electrically connected with the laser emitter 320, and the communicating and processing module 310 controls the laser emitter 320 to generate and emit a stimulating laser beam. For example, the communicating and processing module can be a microcontroller, or a system on chip (SoC) with communicating and processing functions.

The stimulating laser beam is emitted onto a user’s skin to massage the user. Preferably, the stimulating laser beam is emitted onto specific acupuncture points of the user to achieve an effect similar to applying pressure and massaging acupuncture points on the human body. The stimulating laser beam has a wavelength of a specific waveband, such as the wavelength between the waveband of red and near-infrared light (600nm~1000nm). The stimulating laser beam is emitted onto and stimulates the user’s acupuncture points to achieve the massaging effect.

The control device 200 comprises a processing module 210, a communicating module 220, a memory module 230, a display module 240, and an input module 250, wherein the processing module 210 is respectively and electrically connected with the communicating module 220, the memory module 230, the display module 240, and the input module 250, wherein the processing module 20 can be a central processing unit (CPU) of the smartphone (the tablet computer), the communicating module 200 can be an antenna of the smartphone (the tablet computer), and the memory module 230 can be a storage of the smartphone (the tablet computer).

The processing module 210 is connected with the communicating and processing module 310 of the laser device 300 through the communicating module 220. The processing module 210 can also be connected with a network through the communicating module 220, so as to further connect to the server 100 through the network. The memory module 230 stores at least one massage setting parameter. The display module 240 is configured to display information to the user using the control device 200, and the input module 250 is used for the user using the control device 200 to operate and input information into the processing module 210 of the control device 200.

When the processing module 210 receives a physiological data information from the input module 250, the processing module 210 transmits the physiological data information to the server 100 through the communicating module 220. When the server 100 receives the physiological data information, the server 100 updates the customized massage setting information according to the physiological data information and the parameter table, and transmits an updated customized massage setting information to the control device 200.

When the processing module 210 receives the updated customized massage setting information transmitted by the server 100 through the communicating module 220, the processing module 210 updates the at least one massage setting parameter according to the customized massage setting information. The processing module 210 controls the laser emitter 320 of the laser device 300 through the communicating module 220 to generate the stimulating laser beam according to the at least one updated massage setting parameter.

The server 100 assists in managing the physiological data information uploaded by the control device 200, and updates the customized massage setting information according to the physiological data information and the parameter table, and transmits the updated customized massage setting information back to the control device 200. Therefore, the user can use the control device 200 to control the laser device 300 to provide a more appropriate and personalized massage. For the control device 200, the processing module 210 updates the at least one massage setting parameter according to the customized massage setting information, and controls the laser emitter 320 of the laser device 300 to generate the stimulating laser beam according to the at least one massage setting parameter.

Referring to FIG. 1, in an embodiment of the present invention, the communicating module 220 of the control device 200 is respectively and wirelessly connected with the server 100 and the communicating and processing module 310 of the laser device 300. For example, the communicating module 220 is wirelessly connected to the network through Wi-Fi and connected to the server 100 through the network, and is wirelessly connected to the communicating and processing module 310 of the laser device 300 through Bluetooth. The laser device 300 can be a laser massager, and the stimulating laser beam generated through the laser emitter 320 of the laser device 300 can be a massage laser beam.

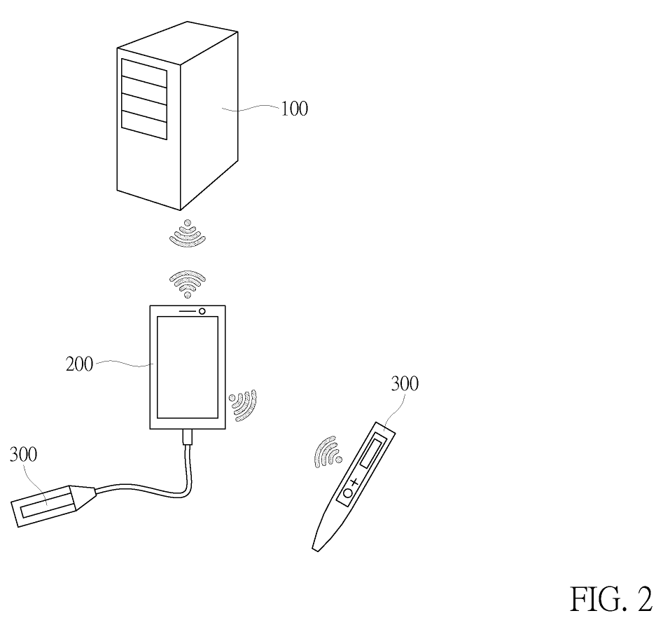

Referring to FIG. 2, in an embodiment, the communicating module 220 of the control device 200 is communicatively connected with the communicating and processing module 310 of the laser device 300 through a port and a wire. For example, the communicating module 200 is electrically connected with the communicating and processing module 310 of the laser device 300 through a universal serial bus (USB) connector or a lightning connector. The laser device 300, connected with the control device 200 through the USB connector or the lightning connector, can communicate with the control device 200 while being charged through the control device 200. The control device 200 can be connected with multiple said laser devices 300, and each laser device 300 connected to the control device 20 does not need to be of the same model. The control device can be a smartphone, a tablet computer, or other network-connected computer devices. In the present embodiment, the control device 200 is the smartphone, and the display module 240 and the input module 250 of the control device 200 are integrated into a touch screen of the smartphone. The processing module 210 of the control device 200 executes an application (APP) to be connected with the server 100.

To ensure an identity of the user using the control device 200, the APP executed by the control device 200 has a membership system that requires an account to log in. When the user operates the input module 250 to input a log-in information, the control device 200 uploads the log-in information to the server 100. When the server 100 receives the log-in information uploaded through the control device 200, the server 100 determines whether the log-in information matches the user’s information.

When the server 100 determines the log-in information matches the user’s information, the identity of the user using the control device 200 (the smartphone) is thus verified and the server 100 allows the control device 200 to access the data on the server 100. The server 100 therefore allows to receive the physiological data information uploaded by the control device 200.

On the contrary, when the server 100 determines the log-in information does not match the user’s information, the server 100 transmits a command back to the control device 200. The processing module 210 of the control device 200 executes a registration process through the command. In particular, the control device 200 displays a registration page through the display module 240 to invite the user to register and become a member.

Moreover, the server 100 stores a physiological data questionnaire information and a massage reminding time corresponding to the user’s information. Whenever the server 100 determines that the massage reminding time has elapsed, the server 100 transmits a massage reminding message and the physiological data questionnaire information to the control device 200. When the processing module 210 of the control device 200 receives the massage reminding message from the communicating module 220, the processing module 210 controls the display module 240 to display the massage reminding message. When the processing module 210 of the control device 200 receives the physiological data questionnaire information from the communicating module 220, the processing module 210 controls the display module 240 to display a physiological data questionnaire according to the physiological data questionnaire information.

The user using the control device 200 can know a recommended time for a massage to maintain the user’s body health according to the massage reminding message displayed on the display module 240. Before using the laser device 300 for the massage, the user can view the physiological data questionnaire displayed on the display module 240 and operate the input module 250 of the control device 200 to answer the physiological data questionnaire, that is, the user inputs the physiological data information of the user through the input module 250. Thus, the physiological data information received by the processing module 210 from the input module 250 corresponds to an answer data of the physiological data questionnaire.

For example, the physiological data questionnaire displayed on the display module 240 includes questions about the user’s current height, weight, medical history, and other physiological information. The user can input the user’s current height, weight, medical history, and other physiological information according to the questions asked to provide the physiological data information. As aforementioned, the physiological data information will be uploaded into the server 100, wherein the physiological data information includes the user’s latest physiological status. Therefore, the server 100 can customize latest massage parameters for the user according to the user’s latest physiological status and transmits the latest massage parameters to the control device 200. The latest massage parameters can then be loaded into the laser device 300 through the control device 200 to generate the stimulating laser beam.

Specifically, the updated customized massage setting information updated by the server 100 comprises a massage duration information and a massage acupuncture points information. The at least one massage setting parameter stored in the memory module 230 of the control device 200 comprises a massage duration, a massage-acupuncture-point type, a laser frequency parameter, and a duty cycle parameter.

After the server 100 transmits the updated customized massage setting information to the processing module 210 of the control device 200, the processing module 210 of the control device 200 updates the massage duration stored in the memory module 230 according to the massage duration information and updates the massage-acupuncture-point type stored in the memory module 230 according to the massage acupuncture points information. The processing module 210 also updates the laser frequency parameter and the duty cycle parameter according to the customized massage setting information when the processing module 210 updates the massage duration and the massage-acupuncture-point type stored in the memory module 230.

The processing module 210 of the control device 200 loads information of the massage duration, the laser frequency parameter, and the duty cycle parameter to the communicating and processing module 310 of the laser device 300, so that the communicating and processing module 310 controls the laser emitter 320 to generate the stimulating laser beam according to the information of the massage duration, the laser frequency parameter, and the duty cycle parameter. The massage duration is configured to control an emitting duration of the stimulating laser beam emitted by the laser emitter 320. The laser frequency parameter is configured to control a specific frequency or wavelength of the stimulating laser beam emitted by the laser emitter 320. The duty cycle parameter is configured to control a duty cycle of the stimulating laser beam emitted by the laser emitter 320. In addition, when the processing module 210 of the control device 200 controls the laser emitter 320 to generate the stimulating laser beam, the processing module 210 controls the display module 240 of the control device 200 to display an acupuncture point position information corresponding to the massage- acupuncture-point type.

Therefore, the user who uses both the control device 200 and the laser device 300 can view the acupuncture point position information displayed on the display module 240, and thus know exactly where to aim the laser emitter 320 on the body to correctly aim the acupuncture point to start massaging. A major advantage of the present invention is that, while assisting in a management of the user’s massage data, the present invention also allows for a customized adjustment of massage parameters and the acupuncture point position information corresponding to the customized adjustment of massage parameters will be displayed on the display module 240 for the user. Namely, the acupuncture point position information displayed on the display module 240 will reflect the latest, most relevant and most suitable acupuncture point position for the user’s physiological status and needs. Therefore, the user who views the acupuncture point position displayed on the display module 240 can use the laser device 300 effectively, allowing the laser emitter 320 to be precisely aimed at the acupuncture points on the body for massaging. Compared with a conventional laser device, the combined use of the laser device 300 and the control device 200 of the laser stimulation and management system of the present invention allows the user to more effectively use the laser device 300 for self-massaging.

The parameters used by the laser emitter 320, such as the duty cycle for generating the stimulating laser beam, can control energy output of the laser emitter 320 in a unit period. The present invention can intelligently adjust the duty cycle parameter, so that the user receives appropriate energy. For example, for the user who has a weak physiologic, the duty cycle parameter is decreased to stimulate the user with less energy in the unit period to prevent the user from injury. On the contrary, for the user who has a strong physiologic, the duty cycle parameter is increased to stimulate the user with more energy in the unit period to prevent the user from not receiving sufficient stimulation.

Please note that the laser device 30 of the present invention is not a medical device and is merely a massage device used for folk medicine and health preservation. The parameters for generating the stimulating laser beam of the laser device 300 of the present invention can be appropriately modified. Therefore, compared with the conventional laser device, the laser device 300 of the present invention can generate the stimulating laser beam to the user, wherein the stimulating laser beam is more comfortable, more suitable, and more health-preservation enhancing for the user.

In another embodiment of the present invention, the control device 200 further comprises a sound module 260 electrically connected with the processing module 210. The control device 200 is a smartphone. The display module 240 of the control device 200 is a touch screen of the smartphone, the sound module 260 of the control device 200 is a speaker of the smartphone, and the input module 250 of the control device 200 comprises the touch screen and a microphone of the smartphone.

In other words, in the present embodiment, the user can operate the smartphone through voice. When the display module 240 displays the physiological data questionnaire, the processing module 210 of the control device 200 also controls the sound module 260 to read out the physiological data questionnaire for the user to listen to. When the user answers the physiological data questionnaire by voice, the input module 250 generates the physiological data information through sound recording of the microphone and transmits the physiological data information to the processing module 210. When the processing module 210 controls the laser emitter 320 of the laser device 300 through the communicating module 220 to generate the stimulating laser beam, the processing module 210 also controls the sound module 260 to read out an operation instruction (to generate a voice of the operation instruction). For example, the operation instruction read out by the sound module 260 can be the acupuncture point position information. Namely, the control device 200 can instruct the user through voice on how to aim the laser emitter 320 at the user’s acupuncture points for effective self-massage.

In the embodiment, a vibration module 330 (such as a vibration motor) is built in the laser device 300 and electrically connected with the communicating and processing module 310. The at least one massage setting parameter updated according to the customized massage setting data through the processing module of the control device 200 also comprises a vibration frequency parameter. The processing module 210 of the control device 200 also loads an updated vibration frequency parameter to the communicating and processing module 310, so that the communicating and processing module 310 uses the updated vibration frequency parameter to control the vibration module 330 to generate a massage vibration. The massage vibration increases blood flow in capillaries of the user’s tense muscles, thereby increasing oxygen saturations of the muscles and accelerating eliminations of lactic acid, thus relaxing a nervous system of the user. Therefore, the laser device 300 of the present invention can provide a variety of customized massage functions for the user by generating the massage vibration through the vibration module 330.

When the processing module 210 of the control device 200 controls the laser emitter 320 of the laser device 300 to generate the stimulating laser beam, the processing module 210 starts recording a usage parameter data in the memory module 230. When the processing module 210 stops controlling the laser device 300 to generate the stimulating laser beam, the processing module stops recording the usage parameter data and transmits the usage parameter data to the server 100 through the communicating module 220. When the server 100 receives the usage parameter data from the control device 200, the server 100 stores the usage parameter data as a usage record and links the usage record with the user’s information. The usage record can serve as a basis for optimization and adjustment of data, laser parameters, etc. in the future. For example, the usage record can serve as the basis for updating the parameter table in the future.

When the server 100 is connected with multiple said control devices 200 and receives multiple usage records of multiple different users through the multiple said control devices 200, the server 100 can collect big data and use the usage records collected from the big data to optimize the parameter table. Thus, when the server 100 subsequently updates the user’s customized massage setting information according to the physiological data information and the parameter table, the server 100 will be able to update and provide the most appropriate and optimized parameters. The parameters can be loaded by the user’s control device 200 into the laser device 300 for use.

For example, the parameter table provides messaging information of acupuncture points in Traditional Chinese Medicine. According to the parameter table stored in the server 100, the user with gastrointestinal discomfort is recommended to massage a Neigua point (PC6), a Zhongwan point (CV12), a Zusanli point (ST36), or a Xiangu point (ST43). However, because of differences in individual physiques, the Neigua point, the Zhongwan point, the Zusanli point, or the Xiangu point may be hard to find for the user. To overcome the issue, the server 100 can use big data containing physiological data from multiple users to an acupuncture point whose location remains relatively constant in the Neigua point, the Zhongwan point, the Zusanli point, and the Xiangu point, and recommend the user to massage the acupuncture point whose location remains relatively constant, increasing a chance of the user to successfully massage the acupuncture point. For example, the acupuncture point whose position remains relatively constant is the Neigua point (that is, for the different user bodies, the Neigua point is usually located in a same spot and is generally easy to find). Therefore, regarding user’s gastrointestinal discomfort, the server 100 computes a result according to the big data, wherein the result is that massaging the Neigua point has the highest chance of achieving a desired effect. The server 100 updates the parameter table according to the result. Whenever the physiological data information uploaded by the control device 200 includes the user has gastrointestinal discomfort, the server 100 updates the customized massage setting information according to the physiological data information and the parameter table, so that the laser device 300 generates laser parameters corresponding to the Neigua point and provides the user with visual and voice instructions to recommend the user aiming the laser device 300 to a position of the Neigua point to stimulate and massage. Therefore, the server 100 can intelligently assist in managing how the user uses the laser device 300, enabling the user to use the laser device successfully and efficiently to improve the user’ healthy.

In another application of the present invention, the user could also be a healthcare worker administering laser stimulation to a patient. The healthcare worker inputs the patient’s information as the physiological data information, and then listens to the recommended acupuncture point provided by the present invention and operates the laser device 300 to apply the laser stimulation to the patient’s acupuncture point with medical knowledge of the healthcare worker. Regardless of application of the preset invention, the laser stimulation and management system of the present invention can intelligently assist in management and provide highly valuable recommendations. The present invention assists operators of the control device 200 and the laser device 300 in efficiently finding locations of the acupuncture points that require massage and generates the stimulating laser beam with appropriate laser parameters.

Furthermore, the user can use the input module 250 of the control device 200 to modify the information corresponding to a member data in the server 100 of the user. When the user using the control device 200 has a system administrator permission, the user can use the input module 250 of the control device 200 to access other internal data in the server 100. That is, when the control device 200 receives an accessing command through the input module 250, the processing module 210 transmits the accessing command to the server 100 through the communicating module 220. When the server 100 receives the accessing command, the server performs corresponding actions on the stored internal data according to the accessing command and transmits back a report message to the control device 200. When the processing module 210 of the control device 200 receives the report message through the communicating module 220, the processing module 210 controls the display module 240 to display the report message.

For example, the accessing command may be a searching command, a modifying command, or a deleting command. The server 100 can search the internal data according to the searching command and transmit back a searching result to the control device 200, so that the display module 240 of the control device 200 displays the searching result as the report message. The server 100 can modify the internal data according to the modifying command and transmit back a modifying result to the control device 200, so that the display module 240 of the control device 200 displays the modifying result as the report message. The server 100 can delete the internal data according to the deleting command and transmit back a deleting result to the control device 200, so that the display module 240 of the control device displays the deleting result as the report message.

The laser stimulation and management system of the present invention is configured to execute a laser stimulation and management method.

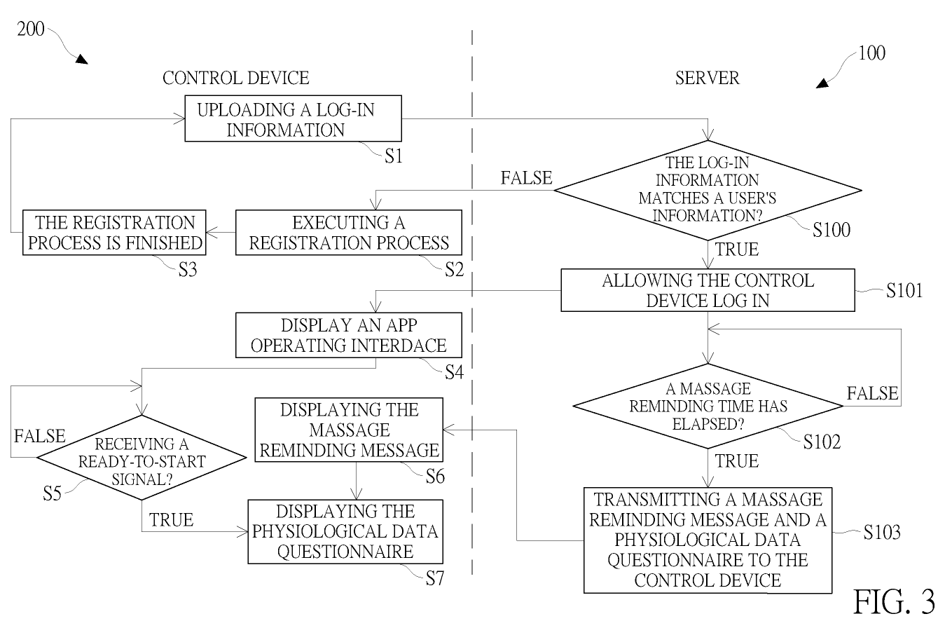

Referring to FIG. 3, in an embodiment, before the control device 200 transmits the physiological data information to the server 100, the laser stimulation and management method comprises the following steps.

The steps executed by the control device 200 comprises:

Step S1: uploading the log-in information to the server 100 inputted from the input module 250;

Step S2: executing a registration process when the log-in information does not match the user’s information;

Step S3: executing the step S1 when the registration process is finished;

Step S4: displaying an APP operating interface when the log-in information matches the user’s information;

Step S5: determining whether receiving a ready-to-start signal through the input module 250; if the ready-to-start signal is not received through the input module 250, the control device 200 executes the step S5; if the ready-to-start signal is received through the input module 250, the control device 200 executes a step S7;

Step S6: controlling the display module 240 to display the massage reminding message when the control device 200 receives the massage reminding message from the server 100;

Step S7: controlling the display module 240 to display the physiological data questionnaire information.

The steps executed by the server 100 comprise:

Step S100: receiving the log-in information from the control device 200 and determining whether the log-in information matches the user’s information; when the log-in information does not match the user’s information, the server 100 controls the control device 200 to execute the registration process;

Step S101: allowing the control device 200 log in the server 100 and controlling the control device 200 to display the APP operating interface when the log-in information matches the user’s information;

Step S102: determining whether the massage reminding time has elapsed; when the massage reminding time has not elapsed, the server 100 executes the step S102;

Step S103: transmitting the massage reminding message and the physiological data questionnaire information to the control device 200 when the massage reminding time has elapsed.

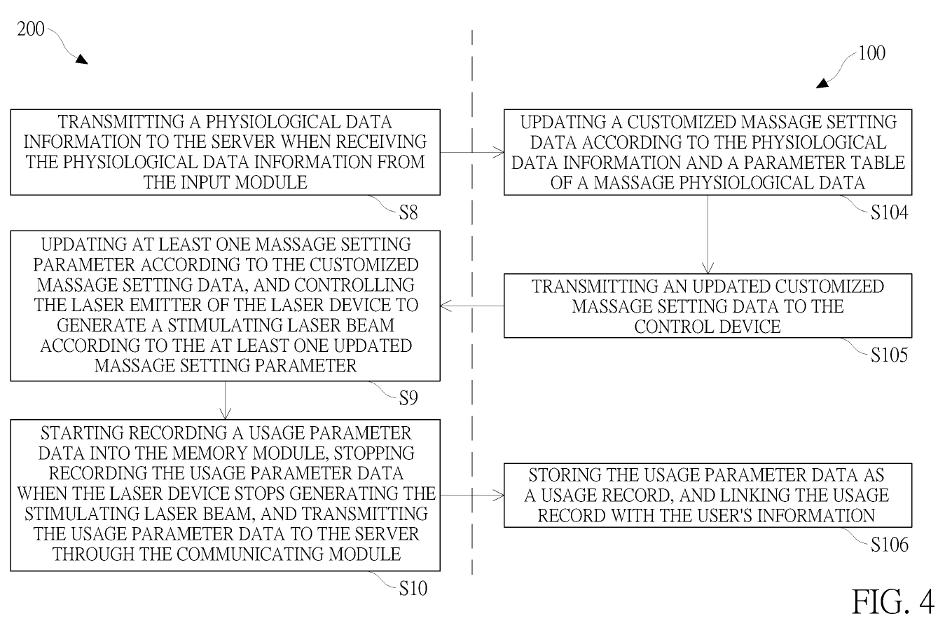

Referring to FIG. 4, in an embodiment, when the control device 200 transmits the physiological data information to the server 100, the laser stimulation and management method comprises the following steps.

The steps executed by the control device 200 comprise:

Step S8: transmitting the physiological data information to the server 100 when the control device 200 receives the physiological data information from the input module 250;

Step S9: updating the at least one massage setting parameter according to the customized massage setting data, and controlling the laser emitter 320 of the laser device 300 to generate the stimulating laser beam according to the at least one updated massage setting parameter;

Step S10: starting recording the usage parameter data into the memory module 230, stopping recording the usage parameter data when the laser device 300 stops generating the stimulating laser beam, and transmitting the usage parameter data to the server 100 through the communicating module 220.

The steps executed by the server 100 comprise:

Step S104: updating the customized massage setting data corresponding to the user’s information according to the physiological data information and the parameter table;

Step S105: transmitting the updated customized massage setting data to the control device 200;

Step S106: storing the usage parameter data transmitted from the control device 200 as the usage record, and linking the usage record with the user’s information.

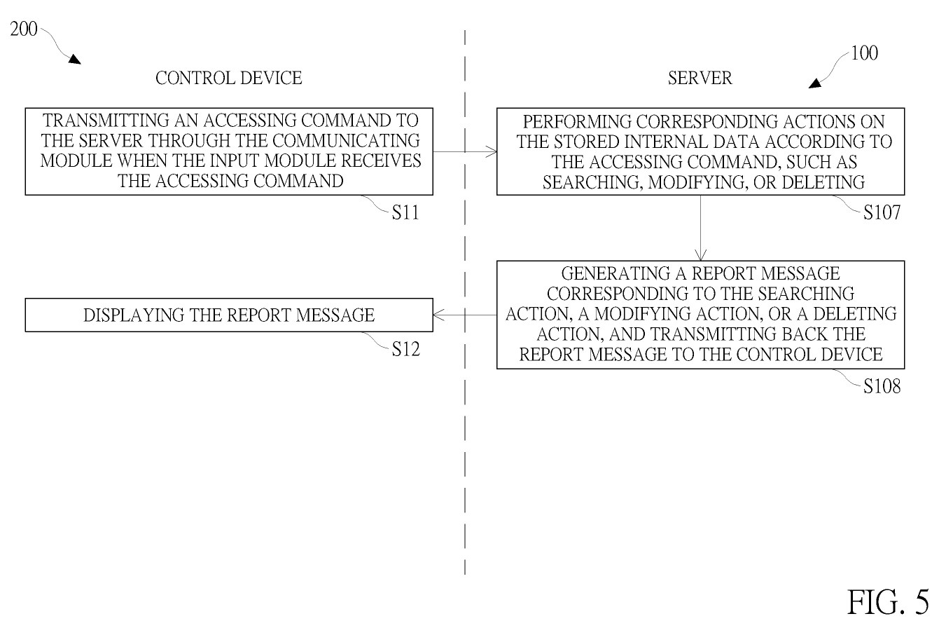

Referring to FIG. 5, in an embodiment of present invention, when the control device 200 accesses the contents of the server 100, the laser stimulation and management method comprises the following steps.

The steps executed by the control device 200 comprise:

Step S11: transmitting the accessing command to the server 100 through the communicating module 220 when the input module 250 receives the accessing command;

Step S12: controlling the display to display the report message when the control device 200 receives the report message.

The steps executed by the server 100 comprise:

Step S107: performing corresponding actions on the stored internal data according to the accessing command, such as searching, modifying, or deleting.

Step S108: generating the report message corresponding to the searching action, a modifying action, or a deleting action, and transmitting back the report message to the control device 200.

What is claimed is:

1. A laser stimulation and management system comprising:
a laser device comprising:
a laser emitter generating and emitting a stimulating laser beam; and
a communicating and processing module electrically connected with the laser emitter;
a server storing a user’s information, a customized massage setting information corresponding to the user’s information, and a parameter table of a massage physiological data corresponding to the user’s information; and
a control device comprising:
a communicating module communicatively connected with the communicating and processing module of the laser device and communicatively connected with the server through a network;
a memory module storing at least one massage setting parameter;
a display module;
an input module; and
a processing module electrically connected with the communicating module, the memory module, the display module, and the input module;
wherein when the processing module receives a physiological data information from the input module, the processing module transmits the physiological data information to the server through the communicating module;
wherein when the server receives the physiological data information, the server updates the customized massage setting information according to the physiological data information and the parameter table, and transmits an updated customized massage setting information to the processing module;
wherein when the processing module receives the updated customized massage setting information from the communicating module, the processing module updates the at least one massage setting parameter according to the customized massage setting information and controls the laser emitter of the laser device through the communicating module to generate the stimulating laser beam according to the at least one updated massage setting parameter;
wherein the customized massage setting information updated by the server comprises a massage acupuncture points information, and the at least one massage setting parameter stored in the memory module of the control device comprises a massage-acupuncture-point type;
wherein after the server transmits the updated customized massage setting information to the processing module, the processing module of the control device updates the massage-acupuncture-point type stored in the memory module according to the massage acupuncture points information;
wherein the processing module of the control device controls the display module to display an acupuncture point position information corresponding to the massage-acupuncture-point type.

2. The laser stimulation and management system as claimed in claim 1, wherein:
the server stores a physiological data questionnaire information and transmits the physiological data questionnaire information to the control device;
when the processing module of the control device receives the physiological data questionnaire information from the communicating module, the processing module controls the display module to display a physiological data questionnaire;
the physiological data information received by the processing module from the input module corresponds to an answer data of the physiological data questionnaire.

3. The laser stimulation and management system as claimed in claim 2, wherein:
the server stores a massage reminding time corresponding to the user’s information;
whenever the server determines that the massage reminding time has elapsed, the server transmits a massage reminding message and the physiological data questionnaire information to the control device;
when the control device receives the massage reminding message, the processing module controls the display module to display the massage reminding message.

4. The laser stimulation and management system as claimed in claim 1, wherein:
the customized massage setting information updated by the server comprises a massage duration information, and the at least one massage setting parameter stored in the memory module of the control device comprises a massage duration;
after the server transmits the updated customized massage setting information to the processing module, the processing module of the control device updates the massage duration stored in the memory module according to the massage duration information;
the processing module of the control device controls an emitting duration of the stimulating laser beam emitted by the laser emitter of the laser device according to the massage duration information.

5. The laser stimulation and management system as claimed in claim 4, wherein:
the at least one massage setting parameter stored in the memory module of the control device comprises a laser frequency parameter and a duty cycle parameter;
when the processing module updates the massage duration and the massage-acupuncture-point type stored in the memory module, the processing module also updates the laser frequency parameter and the duty cycle parameter according to the customized massage setting information;
the processing module of the control device controls the laser emitter of the laser device to generate the stimulating laser beam according to the updated laser frequency parameter and the updated duty cycle parameter.

6. The laser stimulation and management system as claimed in claim 1, wherein:
a vibration module is built-in in the laser device and electrically connected with the communicating and processing module;
the at least one massage setting parameter updated according to the customized massage setting data through the processing module of the control device comprises a vibration frequency parameter;
the processing module of the control device controls the vibration module of the laser device to generate a massage vibration according to an updated vibration frequency parameter.

7. The laser stimulation and management system as claimed in claim 1, wherein:
the control device is a smartphone, and the display module and the input module of the control device are integrated into a touch screen;
the processing module of the control device executes an application to be connected with the server;
when the server receives a log-in information uploaded from the control device, the server determines whether the log-in information matches the user’s information;
when the server determines the log-in information matches the user’s information, the server allows the smartphone to access the server and to receive the physiological data information uploaded by the control device.

8. The laser stimulation and management system as claimed in claim 1, wherein:
the control device comprises a sound module electrically connected with the processing module, and the control device is a smartphone;
the display module of the control device is a touch screen, the sound module of the control device is a speaker, and the input module of the control device comprises the touch screen and a microphone;
the input module generates the physiological data information through sound recording of the microphone and transmits the physiological data information to the processing module;
when the processing module controls the laser emitter of the laser device through the communicating module to generate the stimulating laser beam, the processing module controls the sound module to generate a voice of an operation instruction.

9. The laser stimulation and management system as claimed in claim 1, wherein:
when the processing module of the control device controls the laser emitter of the laser device to generate the stimulating laser beam, the processing module starts recording a usage parameter data in the memory module;
when the processing module stops controlling the laser device to generate the stimulating laser beam, the processing module stops recording the usage parameter data and transmits the usage parameter data to the server through the communicating module;
when the server receivers the usage parameter data from the control device, the server stores the usage parameter data as a usage record and links the usage record with the user’s information.

10. The laser stimulation and management system as claimed in claim 1, wherein:
when the control device receives an accessing command through the input module, the processing module transmits the accessing command to the server through the communicating module;
when the server receives the accessing command, the server performs corresponding actions on stored internal data according to the accessing command and transmits back a report message to the control device;
the accessing command is a searching command, a modifying command, or a deleting command.
